(19) United States
(12) Patent Application Publication
(10) Pub. No.: US 2025/0241543 A1
(43) Pub. Date: Jul. 31, 2025

(54) WEARABLE DEVICE

(71) Applicant: GOERTEK INC., Weifang, Shandong (CN)

(72) Inventors: Jiazheng YAN, Weifang, Shandong (CN); Guangzhi SHAO, Weifang, Shandong (CN)

(21) Appl. No.: 18/695,031

(22) PCT Filed: Dec. 15, 2021
(86) PCT No.: PCT/CN2021/138215

§ 371 Date: Mar. 25, 2024

(30) Foreign Application Priority Data
Oct. 28, 2021 (CN) 202111262789.4

(51) Int. Cl. A61B 5/021; A61B 5/00; A61B 5/024

(52) U.S. Cl. CPC A61B 5/021; A61B 5/02438; A61B 5/681; A61B 5/6844; A61B 5/746

(57) ABSTRACT

A wearable device comprises a device body, a strap body and a detection sensor provided on at least one of the device body and the strap body and at least obtain a user's blood pressure data; a shape memory alloy component connected between the device body and the strap body, and pulled the strap body to move toward the device body; a driving component suppling power to the shape memory alloy component according to a target current magnitude; a main controller sending a detection instruction to the driving component and obtaining detection data from the detection sensor. The wearable device supplies power to the shape memory alloy component, so that it uses its own heat to deform and drives the strap body to move. The strap body may apply pressure on the user to meet detection condition, the structure is simple, miniaturization can be achieved, and reliability is high.

The present application claims a priority to the Chinese patent application No. 202111262789.4, entitled “Wearable Device”, filed with the China Patent Office on Oct. 28, 2021, the entire content of which is incorporated herein by reference.

### TECHNICAL FIELD

The disclosure relates to a field of wearable devices, and particularly, to a wearable device.

### BACKGROUND

In recent years, more and more wearable devices have come into public view, and their popularity has become higher and higher, which greatly improves people's quality of life. Moreover, with the improvement of people's living standards, people are paying more and more attention to medical health, and the function of measuring blood pressure has become a commonly used function for people.

In the prior art, a blood pressure measurement device is generally of a split type, wherein a cuff is connected to the measurement device. The cuff is sleeved on the arm of the subject and the blood pressure data is obtained after pressurization. However, in the prior art, the blood pressure measurement device has many shortcomings such as large size and inconvenience to carry.

Therefore, how to implement the blood pressure detection function of the wearable device is a technical problem needed to be solved currently for those skilled in the art.

### SUMMARY

A purpose of the disclosure is to provide a wearable device that not only satisfies the functions of the wearable device itself, but also meets the need of blood pressure detection, which achieves the purpose of miniaturization, has a simple structure and high reliability.

In order to achieve the above purpose, the disclosure provides the following technical solutions:

A wearable device, comprising a device body and a strap body, wherein the wearable device further comprises:
a detection sensor provided on at least one of the device body and the strap body, the detection sensor being configured to at least obtain a user's blood pressure data;
a shape memory alloy component connected between the device body and the strap body, and configured to pull the strap body to move toward the device body;
a driving component configured to supply power to the shape memory alloy component according to a target current magnitude; and
a main controller configured to send a detection instruction to the driving component and obtain detection data from the detection sensor.

Preferably, the driving component comprises a driver circuit and a driver controller, the driver controller is configured to obtain the target current magnitude and control the driver circuit to supply power to the shape memory alloy component according to the target current magnitude.

Preferably, the driving component further comprises a blood pressure controller configured to calculate the target current magnitude according to a preset contraction rate of the shape memory alloy component.

Preferably, the shape memory alloy component comprises at least one shape memory alloy spring.

Preferably, the wearable device further comprises a cable provided between the device body and the strap body, wherein the cable comprises a transmission line connected between the detection sensor and the main controller and a power line connected between the driving component and the shape memory alloy component.

Preferably, a connecting piece is fixed on one side of the strap body close to the device body, and the shape memory alloy component has one end installed on the connecting piece and the other end installed on the device body.

Preferably, the device body is provided with a limiting guide groove having a notch provided with a limiting boss that limits the strap body, and the limiting boss n be engaged with the connecting piece.

Preferably, the wearable device further comprises a distance sensor configured to obtain a distance between the connecting piece and the device body, wherein the main controller is configured to inform indicating information to the user when the distance acquired by the distance sensor is less than a preset distance.

Preferably, the device body is further provided with a heart rate sensor, and the main controller is further configured to send the detection instruction to the driving component when a real-time heart rate data obtained by the heart rate sensor is higher than preset heart rate data.

Preferably, the wearable device further comprises:
an alarm component, the main controller being configured to control the alarm component to send an alarm signal when the detection data detected by the detection sensor is higher than a preset health data; and
a signal transmitting component, the main controller being configured to control the signal transmitting component to send preset information or real-time location information to a target device when the detection data detected by the detection sensor (700) is higher than the preset health data.

The wearable device according to the disclosure comprises a device body and a strap body, and the wearable device further comprises: a detection sensor provided on at least one of the device body and the strap body, the detection sensor is configured to at least obtain a user's blood pressure data; a shape memory alloy component connected between the device body and the strap body, and configured to pull the strap body to move toward the device body; a driving component configured to supply power to the shape memory alloy component according to a target current magnitude; and a main controller configured to send a detection instruction to the driving component and obtain detection data from the detection sensor. The wearable device according to the disclosure adopts a principle that the shape memory alloy components may deform as the temperature changes and supplies power to the shape memory alloy component, so that it uses its own heat to deform and drives the strap body to move. The strap body can apply pressure on the user to meet the detection condition of the detection sensor, thereby at least achieving the detection of blood pressure. Therefore, the wearable device can realize the blood pressure detection function, has a simple structure, can be miniaturized, and has high reliability.

In a preferred embodiment, the device body is also provided with the heart rate sensor; the main controller is further configured to send a detection instruction to the driving component when the real-time heart rate data acquired by the heart rate sensor is higher than the preset heart rate data. Through the above settings, the user's crisis state can be detected and after the heart rate sensor detects an abnormal heart beat of the user, the driving component may receive the detection instruction, and the blood pressure detection can be automatically performed.

### BRIEF DESCRIPTION OF DRAWINGS

In order to explain the technical solutions in the embodiments or the prior art of the disclosure more clearly, the drawings needed to be used in the description of the embodiments or the prior art will be briefly introduced below. Obviously, the drawings in the following description are only part of the drawings of the disclosure and for those of ordinary skill in the art, other drawings can be obtained based on the provided drawings without exerting creative efforts.

FIG. 1 is a schematic diagram of the overall structure of a specific implementation of the wearable device provided by the embodiment of the disclosure;

FIG. 2 is a schematic diagram of a front structure of the wearable device shown in FIG. 1;

FIG. 3 is a schematic diagram of a rear structure of the wearable device shown in FIG. 1;

FIG. 4 is a schematic diagram of a side structure of the wearable device shown in FIG. 1;

FIG. 5 is a schematic diagram of a structure of the shape memory alloy component in the wearable device shown in FIG. 1 in a relaxed state;

FIG. 6 is a schematic diagram of a structure of the shape memory alloy component in the wearable device shown in FIG. 1 in a contracted state; and

FIG. 7 is a schematic diagram of a cross-sectional structure of the wearable device shown in FIG. 1,

Wherein: device body—100, limiting guide groove—101, heart rate sensor—102, charging PIN—103, strap body—200, connecting piece—300, shape memory alloy component—400, cable—500, screen—600, and detection sensor—700.

### DETAILED DESCRIPTIONS

The technical solutions in the embodiments of the disclosure will be described below with reference to the drawings in the embodiments of the disclosure. Obviously, the described embodiments are only some of the embodiments of the disclosure, rather than all the embodiments. Based on the embodiments in the disclosure, all other embodiments obtained by those of ordinary skill in the art without creative efforts fall within the scope of protection of the disclosure.

The core of the disclosure is to provide a wearable device that can realize the demand for blood pressure detection without affecting the function of the wearable device itself, and that has a simple structure and high reliability.

In order to enable those skilled in the art to better understand the solution of the disclosure, the disclosure will be further described in detail below in conjunction with the drawings and specific embodiments.

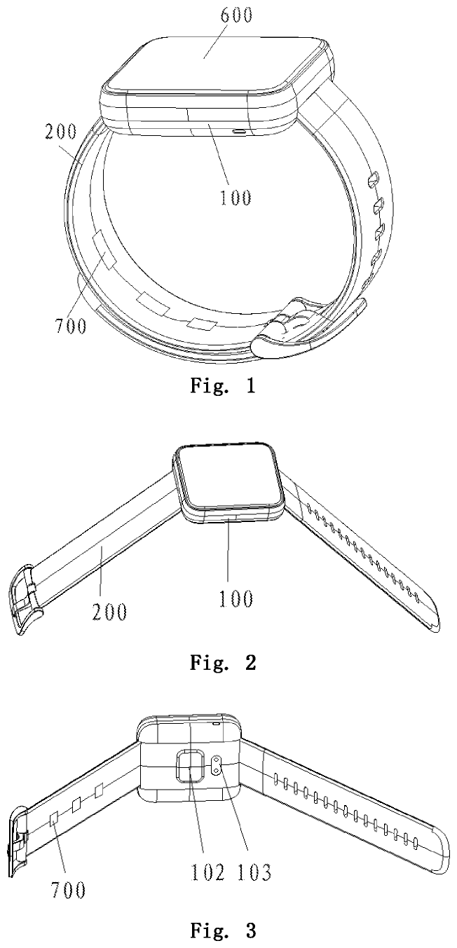

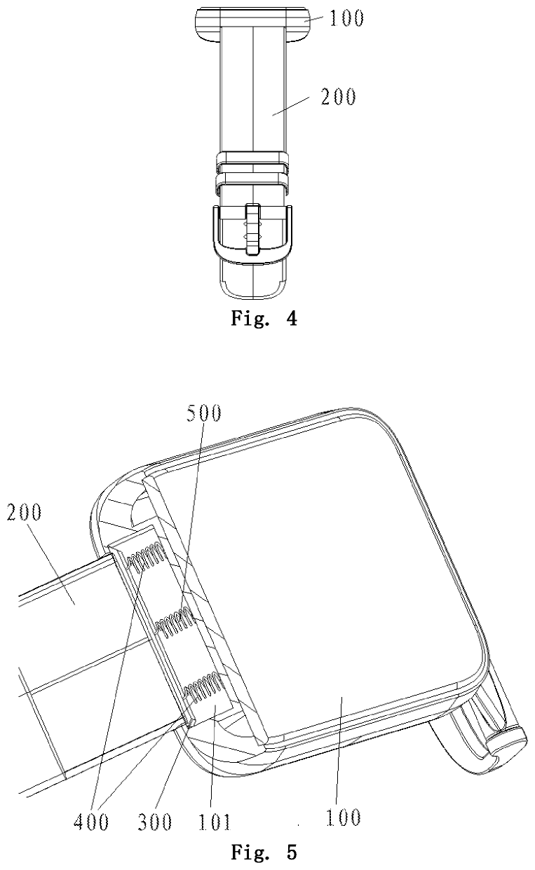

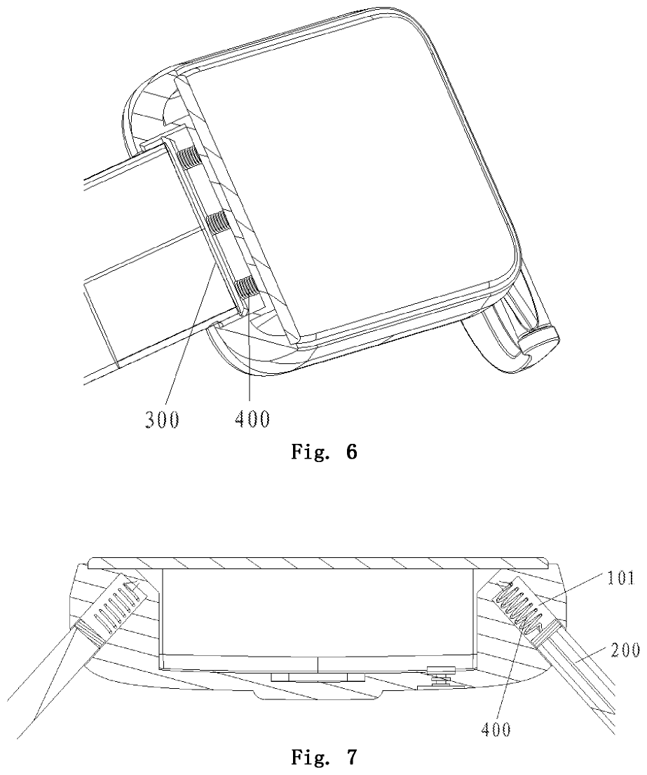

Please refer to FIGS. 1 to 7. FIG. 1 is a schematic diagram of the overall structure of a specific implementation of the wearable device provided by the embodiment of the disclosure; FIG. 2 is a schematic diagram of a front structure of the wearable device shown in FIG. 1; FIG. 3 is a schematic diagram of a rear structure of the wearable device shown in FIG. 1; FIG. 4 is a schematic diagram of a side structure of the wearable device shown in FIG. 1; FIG. 5 is a schematic diagram of a structure of the shape memory alloy component in the wearable device shown in FIG. 1 in a relaxed state; FIG. 6 is a structural diagram of a structure of the shape memory alloy component in the wearable device shown in FIG. 1 in a contracted state; and FIG. 7 is a schematic diagram of a cross-sectional structure of the wearable device shown in FIG. 1.

In this embodiment, the wearable device comprises a device body 100, a strap body 200, a detection sensor 700, a shape memory alloy component 400, a driving component and a main controller. The wearable device may be a watch, a bracelet or other monitoring devices.

Among them, at least one of the device body 100 and the strap body 200 is provided with a detection sensor 700. The detection sensor 700 is configured to at least obtain the user's blood pressure data. Preferably, the detection sensor 700 is provided on the strap body 200. Specifically, the detection sensor 700 may be a blood pressure sensor capable of acquiring pulse data. The number of detection sensors 700 may be multiple to improve detection accuracy. Further, the strap body 200 is provided with an insert, and the detection sensor 700 may be connected to the insert through injection molding. Furthermore, the strap body 200 is preferably made of soft material, which may be high-strength high-temperature resistance silicone or other materials. Specifically, the strap body 200 comprises a first strap body 200 and a second strap body 200 located on two sides of the device body 100 respectively. The first strap body 200 and the second strap body 200 can be arranged as one longer and the other one shorter. The shape memory alloy component 400 may be installed between the first strap body 200 and the device body 100, and/or between the second strap body 200 and the device body 100.

The shape memory alloy component 400 is connected between the device body 100 and the strap body 200. There is an installation space reserved between the device body 100 and the strap body 200. When the shape memory alloy component 400 is contracted, the strap body 200 can be pulled to move toward the device body 100, thereby tightening the strap body 200.

The driving component is configured to supply power to the shape memory alloy component 400 according to a target current magnitude. Since the shape memory alloy component 400 may generate heat and be contracted after being energized, it will drive the strap body 200 to move toward the device body 100.

The main controller is configured to send a detection instruction to the driving component and obtain the detection data detected by the detection sensor 700; at the same time, the main controller can also determine whether the detection condition is met based on the detection data detected by the detection sensor 700. When the detection condition is met, the main controller may control the driving component to gradually reduce the current to the shape memory alloy component 400, and the shape memory alloy component 400 will be gradually restored.

Further, the driving component comprises a driver circuit and a driver controller, that is, the control of the driving component by the main controller is achieved through data transmission with the driver controller. The driver controller is configured to obtain the target current magnitude and control the driver circuit to supply power to the shape memory alloy component 400 according to the target current magnitude.

Specifically, the driving component also comprises a blood pressure controller, which is configured to calculate the target current magnitude according to the preset contraction rate of the shape memory alloy component 400. Specifically, the preset contraction rate may be set according to the use requirements. For example, in the early stage during the tightening of the strap body 200, detection is not needed, the contraction rate of the shape memory alloy component 400 can be increased, and when the detection condition is about to be reached, the contraction rate of the shape memory alloy component 400 may be reduced to improve detection accuracy. The contraction rate of the shape memory alloy component 400 may be achieved by changing the voltage across both ends of the shape memory alloy component 400, that is, the shape memory alloy component 400 may appropriately adjust the voltage across the both ends according to needs. Therefore, the movement speed of the shape memory alloy component 400 is adjusted; more specifically, this can be achieved by adjusting the duty cycle of the driver controller.

Based on the above embodiments, the shape memory alloy component 400 comprises at least one shape memory alloy spring. Preferably, the number of the shape memory alloy spring is at least two, which are located on both sides along a width direction of the strap body 200, or uniformly distributed along the width direction of the strap body 200 to ensure the stability of pulling the strap body 200.

Based on the above embodiments, the wearable device further comprises a cable 500 provided between the device body 100 and the strap body 200. The cable 500 comprises a transmission line and a power line; the transmission line is connected between the detection sensor 700 and the main controller for transmitting signals. The power line is connected between the driving component and the shape memory alloy component 400 for the transmission of current. Preferably, the cable 500 is disposed to be bent, for example, surrounded in the shape of a spring, to adapt to the movement of the strap body 200, thereby reducing the pulling on the cable 500, and improving reliability. At the same time, the device body 100 is also provided with a charging PIN 103 to facilitate charging of the device body 100.

Based on the above embodiments, a connecting piece 300 is fixed on one side of the strap body 200 close to the device body 100. One end of the shape memory alloy component 400 is installed on the connecting piece 300 and the other end is installed on the device body 100. Preferably, in order to facilitate the installation of the shape memory alloy component 400, the connecting piece 300 is preferably a connecting plate. Further, in order to ensure the strength of the connecting piece 300 as the connecting plate, the connecting piece 300 as the connecting plate is made of metal.

Specifically, one end of the power line is connected to the driver circuit, the other end is connected to the wire on the connecting piece 300, the wire is connected to the shape memory alloy component 400, and the other end of the shape memory alloy component 400 is connected to the driver circuit to form a power supply loop; the wires on the connecting piece 300 and the connecting piece 300 itself should be insulated with respect to each other to avoid the influence of the connecting piece 300 on the power supply circuit. Preferably, the cable 500 is located in the middle of each shape memory alloy spring for easy arrangement.

Based on the above embodiments, the device body 100 is provided with a limiting guide groove 101. As shown in FIG. 7, the connecting piece 300 may move in the limiting guide groove 101. The notch of the limiting guide groove 101 is provided with a limiting boss for limiting the strap body 200, and the limiting boss can be engaged with the connecting piece 300 to prevent the strap body 200 from escaping.

Based on the above embodiments, the wearable device also comprises a distance sensor. The distance sensor is configured to obtain the distance between the connecting piece 300 and the device body 100. The main controller is configured to inform indicating information to the user when the distance obtained by the distance sensor is less than the preset distance. Specifically, the distance sensor is disposed on the device body 100. The distance sensor may measure the position of the connecting piece 300 of the strap body 200 and feed the value back to the main controller. When the distance between the position of the connecting piece 300 and the device body 100 is smaller than the preset distance, it means that the strap body 200 is loose and detection cannot be achieved and the user can be reminded to adjust the strap body 200 through vibration or display on the screen 600.

Based on the above embodiments, the device body 100 is also provided with a heart rate sensor 102. The main controller is also configured to send the detection instruction to the driving component when the real-time heart rate data acquired by the heart rate sensor 102 is higher than preset heart rate data. Through the above settings, the user's crisis state maybe detected and after the heart rate sensor 102 detects the abnormal heart beat of the user, the driving component may receive the detection instruction, and the blood pressure detection may be automatically performed.

Based on the above embodiments, the wearable device also comprises:

an alarm component, wherein the main controller is also configured to control the alarm component to send an alarm signal when the detection data detected by the detection sensor 700 is higher than the preset health data. Specifically, the alarm component may be a motor that vibrates to remind the user; or it may trigger the screen 600 to light up to remind the user.

Based on the above embodiments, the wearable device also comprises:

a signal transmitting component, wherein the main controller is also configured to control the signal transmitting component to send preset information or real-time location information to the target device when the detection data detected by the detection sensor 700 is higher than the preset health data. Specifically, when it matches the crisis state set by the program, the device will sound an alarm and can send information and location to the preset personnel to improve safety.

Based on the above embodiments, the device body 100 is also provided with a screen 600, and the screen 600 may display interactive information such as blood pressure values.

In a specific embodiment, as shown in FIG. 5, when the wearable device is worn normally, the shape memory alloy spring is in a relaxed state, its tensile and compressive force values are very small, and its strap body 200 is connected to the device body 100 through the connecting piece 300. Under the action of the limiting guide groove 101 of the device body 100, the connecting piece 300 may only move along the extension direction of the limiting guide groove 101. When it requires to measure blood pressure or the program set in the MCU main controller requires blood pressure detection, the main controller sends a detection command to the blood pressure controller. After receiving the detection command, the blood pressure controller performs calculations and drives the shape memory alloy spring through the driver controller and driver circuit. Since the shape memory alloy component 400 is a resistive component, it generates heat due to the presence of resistance when current passes through. When the heat reaches a certain temperature, the shape memory alloy component 400 is contracted and drives the connecting piece 300 to move. Due to the connection between the connecting piece 300 and the strap body 200, the strap body 200 also moves. At the same time, the detection sensor 700 located on the strap body 200 operates to detect basic information such as blood pressure and pulse, and transmits the signal to the main controller through the cable 500. When the detection condition is reached, that is, when the detection sensor 700 can obtain data such as blood pressure and pulse, the main controller sends an instruction to gradually reduce the current across the both ends of the shape memory alloy component 400. At this time, the temperature of the shape memory alloy component 400 also gradually decreases and the value of the output force also decreases. After the blood pressure detection is completed, the current magnitude across the both ends of the shape memory alloy spring is 0, and the wearable device also returns to the normal wearing state. The state of the shape memory alloy spring after contraction is as shown in FIG. 6.

When the wearable device is worn on the wrist, its detection sensor 700 is in contact with the skin. When the blood pressure measurement program is executed, that is, when the detection instruction is received, the wearable device may become tighter and tighter on the wrist due to the contraction of the shape memory alloy component 400. The detection sensor 700 will transmit a signal to the main controller in the device body 100. When the highest point of the blood pressure measurement force value is reached, the shape memory alloy component 400 stops moving and maintains. As the operation of the blood pressure measurement program, the current across the both ends of the shape memory alloy component 400 gradually decreases, and the wearable device gradually becomes looser on the wrist until it returns to the normal wearing state.

The wearable device according to the disclosure adopts a principle that the shape memory alloy component 400 may deform as the temperature changes and supplies power to the shape memory alloy component 400, so that it uses its own heat to deform and drives the strap body 200 to move. The strap body 200 may apply pressure on the user to meet the detection condition of the detection sensor 700, thereby at least achieving the detection of blood pressure. Therefore, the wearable device can realize the blood pressure detection function, has a simple structure, can be miniaturized, and has high reliability.

The wearable device according to the disclosure has been introduced in detail above. Specific examples are used herein to illustrate the principles and embodiments of the disclosure. The description of the above embodiments is only configured to help understand the method and the core idea of the disclosure. It should be noted that those skilled in the art can implement several improvements and modifications to the disclosure without departing from the principles of the disclosure, and these improvements and modifications also fall within the scope of the claims of the disclosure.

Each embodiment in this specification is described in a parallel or progressive manner. Each embodiment focuses on its differences from other embodiments. The same or similar parts between the various embodiments can be referred to each other.

It should also be noted that relational terms herein such as first and second are only configured to distinguish one entity or operation from another entity or operation, and do not necessarily require or imply that these entities or operations have such actual relationship or sequence between them. Furthermore, the terms “comprise” “include” or any other variations thereof are intended to cover a non-exclusive inclusion such that a process, method, article, or apparatus that comprises a list of elements comprises not only those elements, but also those not expressly listed other elements, or elements inherent to the process, method, article or equipment. Without further limitation, an element defined by the statement “comprises a . . . ” does not exclude the presence of additional identical elements in a process, method, article, or apparatus that comprises the stated element.

1. A wearable device, comprising a device body and a strap body, wherein the wearable device further comprises:
a detection sensor provided on at least one of the device body and the strap body, the detection sensor being configured to at least obtain a user's blood pressure data;
a shape memory alloy component connected between the device body and the strap body, and configured to pull the strap body to move toward the device body;
a driving component configured to supply power to the shape memory alloy component according to a target current magnitude; and
a main controller configured to send a detection instruction to the driving component and obtain detection data from the detection sensor.

2. The wearable device according to claim 1, wherein the driving component comprises a driver circuit and a driver controller, the driver controller is configured to obtain the target current magnitude and control the driver circuit to supply power to the shape memory alloy component according to the target current magnitude.

3. The wearable device according to claim 2, wherein the driving component further comprises a blood pressure controller configured to calculate the target current magnitude according to a preset contraction rate of the shape memory alloy component.

4. The wearable device according to claim 1, wherein the shape memory alloy component comprises at least one shape memory alloy spring.

5. The wearable device according to claim 1, further comprising a cable provided between the device body and the strap body, wherein the cable comprises a transmission line connected between the detection sensor and the main controller and a power line connected between the driving component and the shape memory alloy component.

6. The wearable device according to claim 1, wherein a connecting piece is fixed on one side of the strap body close to the device body, and the shape memory alloy component has one end installed on the connecting piece and the other end installed on the device body.

7. The wearable device according to claim 6, wherein the device body is provided with a limiting guide groove having a notch provided with a limiting boss that limits the strap body, and the limiting boss can be engaged with the connecting piece.

8. The wearable device according to claim 6, further comprising a distance sensor configured to obtain a distance between the connecting piece and the device body, wherein the main controller is configured to inform indicating information to the user when the distance acquired by the distance sensor is less than a preset distance.

9. The wearable device according to claim 1, wherein the device body is further provided with a heart rate sensor, and the main controller is further configured to send the detection instruction to the driving component when a real-time heart rate data obtained by the heart rate sensor is higher than preset heart rate data.

10. The wearable device according to claim 1, further comprising:
an alarm component, the main controller being configured to control the alarm component to send an alarm signal when the detection data detected by the detection sensor is higher than a preset health data; and
a signal transmitting component, the main controller being configured to control the signal transmitting component to send preset information or real-time location information to a target device when the detection data detected by the detection sensor is higher than the preset health data.
